(19) United States
(12) Patent Application Publication
(10) Pub. No.: US 2013/0110534 A1
(43) Pub. Date: May 2, 2013

(54) Specimen Management System, Arrangement, and Method

(71) Applicant: Mobile Aspects, Inc., Pittsburgh, PA (US)

(72) Inventors: Gino Iasella, Gibsonia, PA (US); Timur P. Sriharto, Monroeville, PA (US); Pribadi Kardono, Monroeville, PA (US)

(73) Assignee: Mobile Aspects, Inc., Pittsburgh, PA (US)

(21) Appl. No.: 13/662,897

(22) Filed: Oct. 29, 2012

Related U.S. Application Data
(60) Provisional application No. 61/552,565, filed on Oct. 28, 2011.

(51) Int. Cl. G06Q 50/22

(52) U.S. Cl. 705/2

(57) ABSTRACT

A specimen management system, including a storage unit having at least one access point to provide access to the storage unit, where the storage unit is at least partially associated with storage unit data and configured to receive and store a specimen, which is associated with an identification device associated with specimen data at least partially associated with the specimen. A local controller generates, receives, and/or transmits at least a portion of the storage unit data and/or the specimen data, and the controller initiates at least one action based at least partially on the storage unit data and/or the specimen data. A computer-implemented method for tracking at least one specimen in a tracking system is also disclosed.

### CROSS REFERENCE TO RELATED APPLICATIONS

This application claims benefit of priority from U.S. Provisional Patent Application No. 61/552,565, filed Oct. 28, 2011, which is incorporated by reference in its entirety.

### BACKGROUND OF THE INVENTION

1. Field of the Invention

The present invention relates generally to inventory management methods and systems, and in particular, to a system, arrangement, and method for tracking and managing specimens in hospitals and other healthcare settings, e.g., from the point of collection and/or storage through the point of testing and notice of the results.

2. Description of the Related Art

Presently in the health care industry, there is an emphasis on tracking the results of testing of various specimens, e.g., samples of tissue, blood, urine, and the like. Most existing systems directed to specimen management are related specifically to ensuring no tracking or identification errors occur in the testing laboratory, and are often implemented at hospital pathology/testing departments and larger commercial testing companies. However, there are significant drawbacks and deficiencies existing in these known systems, including, but not limited to: (1) the inability to track information about the specimen before it shows up in the testing environment; (2) the loss of a specimen as it is moved from a point-of-collection to point-of-testing; (3) significant delays that arise from the time of collection to the time of testing; (4) the existence of poor record keeping from the point-of-collection through the point-of-testing, and hand-offs in between; (5) inaccurate identification of the specimen and/or patient, due to handwritten labeling methods; (6) the existence of poor documentation of a “cold chain” (i.e., specimens that are required to be kept cold); and/or (7) insufficient or nonexistent notifications to the person that requested the test regarding the status and/or results relating to the requested test. Further, in such existing systems, specimen samples are often lost before they get to the point-of-testing, rendering any system to reduce errors in the testing lab useless.

In hospitals today, the chain of events surrounding the collection of specimens, retrieval by support staff, transport to testing facilities (inside and outside of the hospital), and subsequent notice back to the requesting clinician is extremely inefficient and includes poor documentation. In most cases, the only documentation of the sample exists on the sample itself until it arrives at the testing lab, where only then is information entered into a computer system.

Still further, the chain of events surrounding the roundtrip of the specimen is currently manual and prone to several communication errors and time lapses. In these existing systems, a clinician from the department collecting the sample (often an operating room nurse, endocrinologist, or staff in similar hospital departments) will call the pathology/testing department to pick-up the sample. There is no record of when the call was placed, who made the call, who received the call, or who has been dispatched to pick up the sample. Often, a clinician is required to leave a message with the testing department, due to heavy collection volumes. After placing the call, the nurse is normally asked by a physician to call to the pathology department to ask why the sample has not yet been picked up.

When the support staff is dispatched from the specimen testing department, a hand-off occurs from the clinicians in the department who requested the test to the support staff. No record is kept of this hand-off, the people involved or the time. The support staff picking up the specimen may then either go directly back to the testing lab, or go to pick-up other specimens. However, this is poorly tracked by hospital administrators, which often leads to frustration. Clinicians then experience extremely long wait times (many hours or even days) before their results are returned or received. In interviews conducted with clinicians on the subject, they often ask where the support staff goes in between these times. This results in further calls from the clinicians in the department to the testing lab. This makes the situation worse, as the lab staff is taking further calls, thereby taking them away from work and receiving other initial calls to dispatch support staff.

In a worst case scenario, a specimen can be lost. Normally, no one in the chain is sure if it was lost before the hand-off to support staff, during transport, or somewhere in the testing lab. Due to poor labeling and documentation, it then becomes a point of contention between staff and departments. The worst affected by this scenario is the patient, as it may have been a difficult sample to obtain, and patient safety may be at stake. Even if it is a simple sample to obtain, it causes tremendous loss of credibility in the health system.

Finally, after the specimen is taken to the testing lab, the testing can occur. The existing systems only begin the tracking process when the specimen enters the lab environment—to ensure that there are no errors in the testing procedures. However, after the results are ready, someone from the testing lab must manually call or e-mail the requesting clinician, and/or the requesting clinician must constantly check the computer system to see if the results are ready. No notification is automatically sent to the clinician. This causes clinicians to constantly call the testing labs to see if the result is ready, if it has been received, if the test has started, etc. A large amount of extra work is created since there is no information about the specimen once it leaves the department of the requesting clinician.

Existing systems represent ineffective, inaccurate, and/or incomplete processes to identify and track specimens throughout their lifecycle, and provide minimal or no information to the managers of any “Continuous Improvement” process. For example, these systems do not determine how long it takes from the time a specimen is collected to the time it is retrieved, to the time it is taken to the testing labs, to the time the testing result is ready. Hence, these existing systems do not effectively track or manage specimens from the point-of-collection through the point-of-notice to the requesting clinician.

Accordingly, there is a need in the art for improved specimen tracking systems, which are effective and efficient in the monitoring, tracking, and communication of data and information directed to a specified specimen throughout its effective lifecycle, i.e., from the point-of-collection through the point-of-testing, with the provision of notice of results back to the requesting clinicians. Therefore, there is a need in the art for a specimen management system, arrangement, and method that provides improved specimen tracking throughout all stages of collection, delivery, testing, and results.

### SUMMARY OF THE INVENTION

Generally, provided is a specimen management system, arrangement, and method that address or overcome certain drawbacks and/or deficiencies in existing specimen management and healthcare systems. Preferably, provided is a specimen management system, arrangement, and method that facilitate accurate and effective tracking of specimens throughout the various stages of the specimen lifecycle.

Accordingly, and in one preferred and non-limiting embodiment, provided is a specimen management system, including at least one storage unit including an at least partially enclosed structure having at least one access point to provide access to the at least one storage unit. The at least one storage unit is at least partially associated with storage unit data and configured to receive and store at least one specimen, which is associated with at least one identification device configured or programmed with specimen data at least partially associated with the at least one specimen. At least one local controller is programmed or configured to generate, receive, and/or transmit at least a portion of at least one of the storage unit data and the specimen data. Further, this at least one local controller is programmed or configured to initiate at least one action based at least partially on at least one of the storage unit data and the specimen data.

In another preferred and non-limiting embodiment, provided is a specimen management system, including at least one storage unit including an at least partially enclosed structure having at least one access point configured to provide access to the at least one storage unit. The at least one storage unit is at least partially associated with storage unit data and configured to receive and store at least one specimen, which is associated with at least one identification device configured or programmed with specimen data at least partially associated with the at least one specimen. The system also includes at least one networked computer in direct or indirect communication with the at least one storage unit and configured to receive at least a portion of at least one of the storage unit data and the specimen data, and the at least one networked computer is programmed or configured to initiate at least one action based at least partially on at least one of the storage unit data and the specimen data.

In a still further preferred and non-limiting embodiment, provided is a computer-implemented method for tracking at least one specimen in a tracking system, including: on at least one computer having a storage medium having stored thereon program instructions, which, when executed by a processor of the computer, cause the processor to: detect the entry of the at least one specimen into the tracking system; determine specimen data at least partially associated with the at least one specimen, wherein the specimen data at least partially comprises at least one of the following: patient data, time data, specimen collection data, specimen purpose data, specimen collector data, specimen collection location data, health care provider data, laboratory data, procedure data, test data, body data, surgery data, storage unit identification data, location data, custody data, results data, compliance data, inventory data, authorization data, access data, user data, status data, alert data, or any combination thereof; and based at least partially on at least a portion of the specimen data, generate, receive, and/or transmit at least one of the following: at least a portion of the specimen data, message data, e-mail data, text data, voice data, status data, alert data, or any combination thereof.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a schematic view of a computer and network infrastructure according to the prior art;

FIG. 2 is a schematic view of one embodiment of specimen management system, arrangement, and method according to the principles of the present invention;

FIG. 3 is a schematic view of one embodiment of a storage unit for use in a specimen management system, arrangement, and method according to the principles of the present invention;

FIG. 4 is a schematic view of one embodiment of an identification device unit for use in a specimen management system, arrangement, and method according to the principles of the present invention; and

FIG. 5 is a schematic view of another embodiment of a storage unit for use in a specimen management system, arrangement, and method according to the principles of the present invention;

### DETAILED DESCRIPTION OF THE PREFERRED EMBODIMENTS

For purposes of the description hereinafter, the terms “end”, “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that may be wired and/or wireless in nature. Additionally, two units or components may be in communication with each other even though the data transmitted may be modified, processed, and/or routed between the first and second unit or component. For example, a first unit may be in communication with a second unit even though the first unit passively receives data, and does not actively transmit data to the second unit. As another example, a first unit may be in communication with a second unit if an intermediary unit processes data from one unit and transmits processed data to the second unit. It will be appreciated that numerous other arrangements are possible. The components or units may be directly connected to each other or may be connected through one or more other devices or components. The various coupling components for the devices can include but are not limited to the Internet, a wireless network, a conventional wire cable, an optical cable or connection through air, water or any other medium that conducts signals, and any other coupling device or medium.

Various aspects of the invention described herein may be applied to any of the particular applications set forth below or in any other type of specimen management or tracking setting or system. Further, the invention may be applied as a stand-alone method or system, or as part of an integrated hospital or healthcare system. It should be understood that different aspects of the invention can be appreciated individually, collectively, or in combination with each other.

Hereinafter, aspects of this invention are described in terms of functional block components, optional selections, and various processing steps. Such functional blocks may be realized by any number of hardware and/or software components configured to perform to specified functions. For example, the invention may employ various integrated circuit components (e.g., memory elements, processing elements, logic elements, look-up tables, and the like), which may carry out a variety of functions under the control of one or more microprocessors or other control devices. Similarly, the software components of this invention may be implemented with any programming or scripting languages such as C, C#, C++, Java, assembler, extensible markup language (XML), extensible stylesheet transformations (XSLT), with the various algorithms being implemented with any combination of data structures, objects, processes, routines, or other programming elements.

Further, it should be noted that this invention may employ any number of conventional techniques for data transmission, signaling, data processing, network control, and the like. In addition, many applications of the present invention could be formulated. The exemplary network disclosed herein may include any system for exchanging data or transacting business, such as the Internet, an intranet, an extranet, WAN, LAN, satellite or cellular communication networks, and/or the like. The terms “Internet” or “network”, as used herein, may refer to the Internet, any replacement, competitor or successor to the Internet, or any public or private internetwork, intranet or extranet that is based upon open or proprietary protocols. Specific information related to the protocols, standards, and application software used in connection with the Internet may not be discussed herein.

Where required, a system user may interact with the system to complete a transaction via any input device or user interface, such as presses or gestures on a touch-screen, user actions that cause a change in readings obtained from sensors, keypad presses, and so on. Similarly, this invention could be used with any kind of smartphone (e.g., Apple iPhone, BlackBerry), handheld computer (e.g., Apple iPad) or used with any type of personal computer, network computer, workstation, minicomputer, mainframe or the like running any operating system, such as any version of Android, Linux, Windows, Windows NT, Windows 2000, Windows XP, MacOS, UNIX, Solaris, iOS or the like. The invention could be implemented using one or more of the following communication protocols: TCP/IP, X.25, SNA, AppleTalk, SCSI, NetBIOS, OSI, GSM, or any number of communication protocols. Moreover, the system contemplates the use, sale, or distribution of any goods, services, or information over any network having similar functionality described herein. A variety of conventional communications media and protocols may be used for the data links. For example, data links may be an Internet Service Provider (ISP) configured to facilitate communications over a local loop as is typically used in connection with standard modem communication, cable modem, dish networks, ISDN, DSL lines, GSM, G4/LTE, WDMCA, or any wireless communication media.

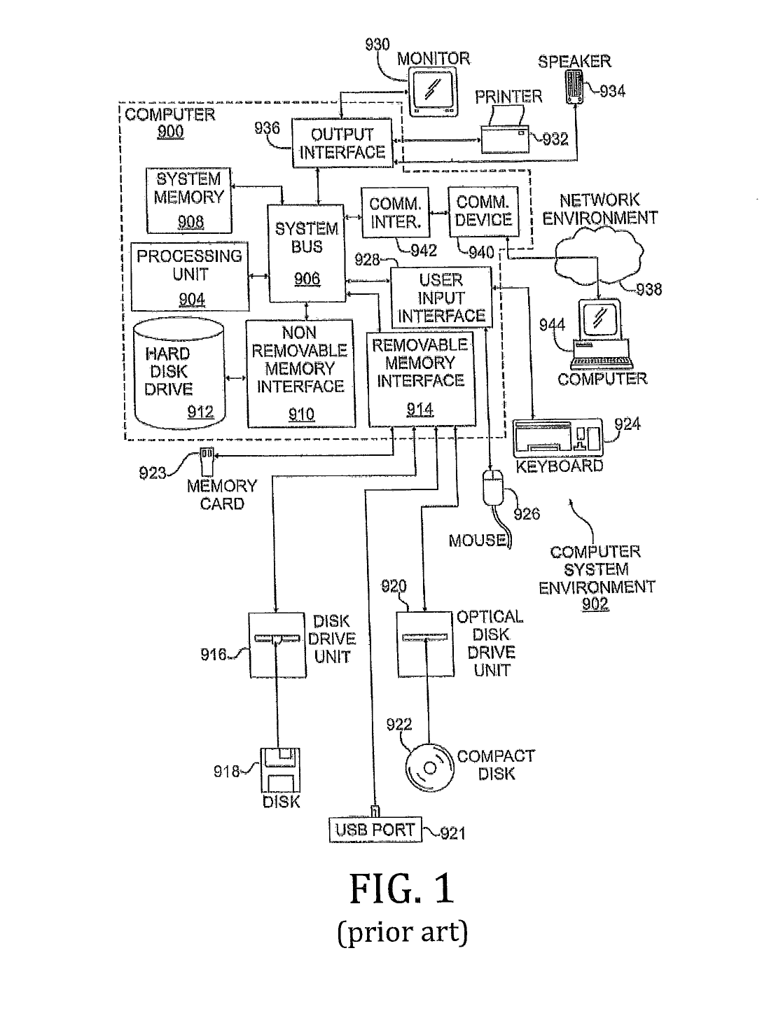

Still further, and discussed hereinafter, it is to be recognized that some or all of the functions, aspects, features, and instances of the present invention may be implemented on a variety of computing devices and systems, wherein these computing devices include the appropriate processing mechanisms and computer-readable media for storing and executing computer-readable instructions, such as programming instructions, code, and the like. As shown in FIG. 1, personal computers 900, 944, in a computing system environment 902 are provided. This computing system environment 902 may include, but is not limited to, at least one computer 900 having certain components for appropriate operation, execution of code, and creation and communication of data. For example, the computer 900 includes a processing unit 904 (typically referred to as a central processing unit or CPU) that serves to execute computer-based instructions received in the appropriate data form and format. Further, this processing unit 904 may be in the form of multiple processors executing code in series, in parallel, or in any other manner for appropriate implementation of the computer-based instructions.

In order to facilitate appropriate data communication and processing information between the various components of the computer 900, a system bus 906 is used. The system bus 906 may be any of several types of bus structures, including a memory bus or memory controller, a peripheral bus, or a local bus using any of a variety of bus architectures. In particular, the system bus 906 facilitates data and information communication between the various components (whether internal or external to the computer 900) through a variety of interfaces, as discussed hereinafter.

The computer 900 may include a variety of discrete computer-readable media components. For example, this computer-readable media may include any media that can be accessed by the computer 900, such as volatile media, non-volatile media, removable media, non-removable media, etc. As a further example, this computer-readable media may include computer storage media, such as media implemented in any method or technology for storage of information, such as computer-readable instructions, data structures, program modules, or other data, random access memory (RAM), read only memory (ROM), electrically erasable programmable read only memory (EEPROM), flash memory, or other memory technology, CD-ROM, digital versatile disks (DVDs), or other optical disk storage, magnetic cassettes, magnetic tape, magnetic disk storage, or other magnetic storage devices, or any other medium which can be used to store the desired information and which can be accessed by the computer 900. Further, this computer-readable media may include communications media, such as computer-readable instructions, data structures, program modules, or other data in other transport mechanisms and include any information delivery media, wired media (such as a wired network and a direct-wired connection), and wireless media. Computer-readable media may include all machine-readable media with the sole exception of transitory, propagating signals. Of course, combinations of any of the above should also be included within the scope of computer-readable media.

The computer 900 further includes a system memory 908 with computer storage media in the form of volatile and non-volatile memory, such as ROM and RAM. A basic input/output system (BIOS) with appropriate computer-based routines assists in transferring information between components within the computer 900 and is normally stored in ROM. The RAM portion of the system memory 908 typically contains data and program modules that are immediately accessible to or presently being operated on by processing unit 904, e.g., an operating system, application programming interfaces, application programs, program modules, program data, and other instruction-based computer-readable codes.

With continued reference to FIG. 1, the computer 900 may also include other removable or non-removable, volatile or non-volatile computer storage media products. For example, the computer 900 may include a non-removable memory interface 910 that communicates with and controls a hard disk drive 912, i.e., a non-removable, non-volatile magnetic medium; and a removable, non-volatile memory interface 914 that communicates with and controls a magnetic disk drive unit 916 (which reads from and writes to a removable, non-volatile magnetic disk 918), an optical disk drive unit 920 (which reads from and writes to a removable, non-volatile optical disk 922, such as a CD ROM), a Universal Serial Bus (USB) port 921 for use in connection with a removable memory card, etc. However, it is envisioned that other removable or non-removable, volatile or non-volatile computer storage media can be used in the exemplary computing system environment 900, including, but not limited to, magnetic tape cassettes, DVDs, digital video tape, solid state RAM, solid state ROM, etc. These various removable or non-removable, volatile or non-volatile magnetic media are in communication with the processing unit 904 and other components of the computer 900 via the system bus 906. The drives and their associated computer storage media discussed above and illustrated in FIG. 1 provide storage of operating systems, computer-readable instructions, application programs, data structures, program modules, program data, and other instruction-based computer-readable code for the computer 900 (whether duplicative or not of this information and data in the system memory 908).

A user may enter commands, information, and data into the computer 900 through certain attachable or operable input devices, such as a keyboard 924, a mouse 926, etc., via a user input interface 928. Of course, a variety of such input devices may be used, e.g., a microphone, a trackball, a joystick, a touchpad, a touch-screen, a scanner, etc., including any arrangement that facilitates the input of data, and information to the computer 900 from an outside source. As discussed, these and other input devices are often connected to the processing unit 904 through the user input interface 928 coupled to the system bus 906, but may be connected by other interface and bus structures, such as a parallel port, game port, or a universal serial bus (USB). Still further, data and information can be presented or provided to a user in an intelligible form or format through certain output devices, such as a monitor 930 (to visually display this information and data in electronic form), a printer 932 (to physically display this information and data in print form), a speaker 934 (to audibly present this information and data in audible form), etc. All of these devices are in communication with the computer 900 through an output interface 936 coupled to the system bus 906. It is envisioned that any such peripheral output devices be used to provide information and data to the user.

The computer 900 may operate in a network environment 938 through the use of a communications device 940, which is integral to the computer or remote therefrom. This communications device 940 is operable by and in communication with the other components of the computer 900 through a communications interface 942. Using such an arrangement, the computer 900 may connect with or otherwise communicate with one or more remote computers, such as a remote computer 944, which may be a personal computer, a server, a router, a network personal computer, a peer device, or other common network nodes, and typically includes many or all of the components described above in connection with the computer 900. Using appropriate communication devices 940, e.g., a modem, a network interface or adapter, etc., the computer 900 may operate within and communicate through a local area network (LAN) and a wide area network (WAN), but may also include other networks such as a virtual private network (VPN), an office network, an enterprise network, an intranet, the Internet, etc. It will be appreciated that the network connections shown are exemplary and other means of establishing a communications link between the computers 900, 944 may be used.

As used herein, the computer 900 includes or is operable to execute appropriate custom-designed or conventional software to perform and implement the processing steps of the method and system of the present invention, thereby forming a specialized and particular computing system. Accordingly, the presently-invented method and system may include one or more computers 900 or similar computing devices having a computer-readable storage medium capable of storing computer-readable program code or instructions that cause the processing unit 904 to execute, configure, or otherwise implement the methods, processes, and transformational data manipulations discussed hereinafter in connection with the present invention. Still further, the computer 900 may be in the form of a smartphone, a tablet computer, a personal computer, a personal digital assistant, a portable computer, a laptop, a palmtop, a mobile device, a mobile telephone, a server, or any other type of computing device having the necessary processing hardware to appropriately process data to effectively implement the presently-invented computer-implemented method and system.

Computer 944 represents one or more work stations appearing outside the local network and users machines. The users interact with computer 900, which can be an exchange system of logically integrated components including a database server and web server. In addition, secure exchange can take place through the Internet using secure www. An e-mail server can reside on system computer 900 or a component thereof. Electronic data interchanges can be transacted through networks connecting computer 900 and computer 944. Third parties represented by computer 944 can connect using EDI or www, but other protocols known to one skilled in the art to connect computers could be used.

The exchange system can be a typical web server running a process to respond to HTTP requests from remote browsers on computer 944. Through HTTP, the exchange system can provide the user interface graphics. It will be apparent to one skilled in the relevant art(s) that the system may utilize databases physically located on one or more computers which may or may not be the same as their respective servers. For example, programming software on computer 900 can control a database physically stored on a separate processor of the network or otherwise.

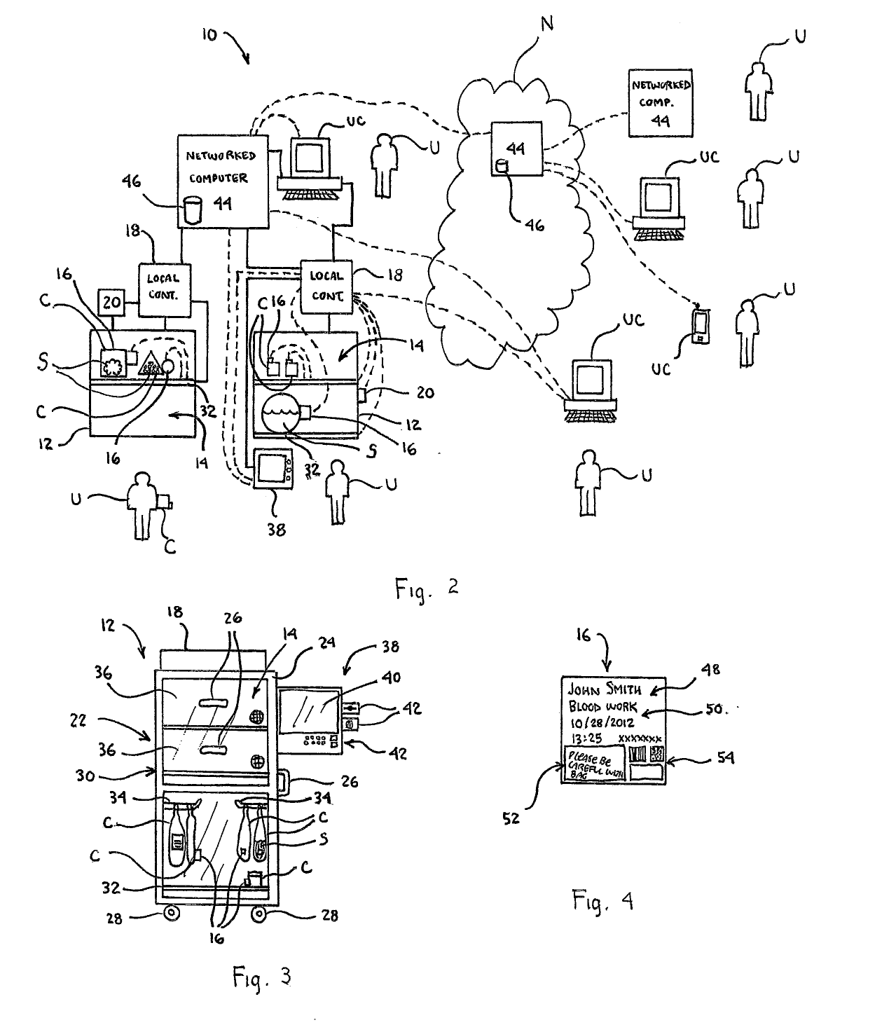

The present invention is directed to a specimen management system, arrangement, and method, which are illustrated in various preferred and non-limiting embodiments in FIGS. 2-5.

In one preferred and non-limiting embodiment, and as illustrated in FIG. 2, the specimen management system 10 includes one (and normally multiple) storage units 12 that are formed as an at least partially enclosed structure with at least one access point 14. The storage unit 12 may be in the form of a cabinet, an enclosed box or storage area, a refrigerator, a freezer, a heating unit, or any other container or containment structure. For example, the storage unit 12 may be in form of or include any of the functions and components described in U.S. patent application Ser. No. 13/158,827 (Publication No. US-2011-0285536-A1) and U.S. patent application Ser. No. 12/607,732 (Publication No. US-2010-0191049-A1), each of which is incorporated herein by reference in its entirety. Accordingly, the access point 14 may also take a variety of forms, such as a door, a drawer, a sliding structure, or any other suitable structure or arrangement for permitting access to the internal area of the storage unit 12. In addition, and as discussed in more detail hereinafter, the storage unit 12 is at least partially associated with storage unit data. Still further, the storage unit 12 may be a specially-manufactured or—configured unit, an existing unit, a modified unit, or any combination thereof.

With continued reference to FIG. 2, the storage unit 12 is configured or arranged to receive and store (e.g., support) at least one (and preferably multiple) specimens S. Further, an identification device 16 is associated with each specimen S, or a grouping of selected specimens S, and is associated, configured, programmed, and/or pre-programmed with specimen data that is at least partially associated with the specimen S. The specimens S may take a variety of forms, including, but not limited to, tissue, blood, urine, biopsy material, body parts, and the like. Accordingly, and in one preferred and non-limiting embodiment, the specimen S is deposited in or stored in a container C, which may be stored in a large container, if required. Accordingly, the identification device 16 would be attached to or associated with the container C storing or containing the specimen S. A variety of sizes and shapes of containers C are envisioned. Accordingly, a corresponding variety of shapes and sizes of identification devices 16 may also be used. In one preferred and non-limiting embodiment, the identification device 16 is a radio frequency identification tag that is attached to at least a portion of the container C. This tag may take a variety of forms, as discussed hereinafter.

As illustrated in the preferred and non-limiting embodiment of FIG. 2, the specimen management system 10 includes a local controller 18, which is programmed or configured to generate, receive, and/or transmit at least a portion of the storage unit data and specimen data. In addition, this local controller 18 is programmed or configured to initiate at least one action (as discussed hereinafter) based at least partially on the content or nature of the storage unit data and/or the specimen data.

The storage unit data and/or the specimen data may take a variety of forms and at least partially include at least one of the following: patient data, time data, specimen collection data, specimen purpose data, specimen collector data, specimen collection location data, health care provider data, laboratory data, procedure data, test data, body data, surgery data, storage unit identification data, location data, custody data, results data, compliance data, inventory data, authorization data, access data, user data, status data, alert data, or any combination thereof. It is some or all of this data that is used in managing and tracking the specimen S throughout the various stages of its life cycle.

The patient data may include, but is not limited to, any information or data relating to the patient, such as the patient's name, contact information, medical information, diagnostic information, personal information, public information, and the like. The time data may include, but is not limited to, the time a request for a specimen S was made, a time of a call to a person (e.g., a clinician, a doctor, a patient, a lab technician, staff, and the like), a time of specimen S pick-up, a time of specimen S drop-off, a time when a specimen S was deposited in or removed from a storage unit 12, any time during the various stages in the life cycle of the specimen S, a time associated with delivery or transport, a collection time, and/or any other time-based information or data. The specimen collection data may include, but is not limited to, any information and data related to the initial or subsequent collection of one or more specimens S from the patient. The specimen purpose data may include, but is not limited to, any information and data related to the reason, medical or otherwise, as to why the specimen S was obtained from the patient. The specimen collector data may include, but is not limited to, any information and data related to the person who collects the specimen S, the person who receives the specimen S, the person who deposits or removes the specimen S from a storage unit 12, or any other person that has been or will be in custody of the specimen S. The specimen collection location data may include, but is not limited to, any information and data related to the position or location of the specimen S during the various stages of its life cycle.

The health care provider data may include, but is not limited to, any information or data relating to the health care employee and/or health care location. The laboratory data may include, but is not limited to, any information and data related to the laboratory where testing is implemented, or any other functions or features relating to the laboratory environment. The procedure data may include, but is not limited to, any information and data related to the procedure that should be implemented in connection with the specimen S and/or the patient. The test data may include, but is not limited to, any information and data related to the testing of the specimen S. The body data may include, but is not limited to, any information and data related to the location on the patient's body from which the specimen S was obtained, the nature or content of the specimen S, any physiological characteristic of the specimen S, and the like. The surgery data may include, but is not limited to, any information and data related to a surgical procedure or process relating to the specimen S.

The storage unit identification data may include, but is not limited to, any information and data related to the storage unit or any of its components. The location data may include, but is not limited to, any information and data related to the physical location or environment of any of the components of the system 10. The custody data may include, but is not limited to, any information or data relating to the custody and/or chain-of-title of the specimen S. The results data may include, but is not limited to, any information and data related to the testing results or any other analysis directed to the specimen S. The compliance data may include, but is not limited to, any information and data related to compliance with any rules or regulations associated with any aspect of the life cycle of the specimen S. The inventory data may include, but is not limited to, any information and data related to inventory management of the specimen S and/or the storage units 12. The authorization data may include, but is not limited to, any information and data related to the authorization or security information of any of the users U, the authorization to handle or control a specimen S, the authorization to deposit or remove specimens S from a storage unit 12, and the like. Access data may include, but is not limited to, any information or data relating to access at a storage unit 12, such as through the access point 14. User data may include, but is not limited to, any information or data relating to any user U in the system 10. Status data may include, but is not limited to, any information and data related to the status of any component of the system 10, the status of any component of the storage cabinet 12, the status of the container C or specimen S, and the like. Alert data may include, but is not limited to, any information or data that is generated or provided to notify or otherwise provide an indication to any user U in the system 10.

In another preferred and non-limiting embodiment, the storage unit 12 is provided with a security arrangement 20 for providing selective access to the storage unit 12, such as through the access point 14. This security arrangement 20 may take a variety of forms, including, but not limited to, a lock, a manual lock, an electronic lock, a magnetic lock, a latching system, a controlled lock, or any other security device or arrangement that can prevent access to the internal area of the storage unit 12. In one preferred and non-limiting embodiment, the security arrangement 20 is in communication with or otherwise controlled by the local controller 18. In this embodiment, the local controller 18 is programmed or configured to unlock the access point 14, e.g., the door, based upon some user U interaction with the storage unit 12. In this manner, only an authorized user U (or a group or set of authorized users U) have access to the storage unit 12. Of course, it is envisioned that the storage unit 12 may have multiple access points 14, e.g., multiple doors or drawers, and each of these doors or drawers can be locked and/or unlocked based upon the level or content of the user U authorization, e.g., the authorization data. In this manner, a user U may have total or only partial access to various areas or components of the storage unit 12.

In addition, and in another preferred and non-limiting embodiment, the storage unit 12 is in the form of or is in communication with a preexisting unit, such as an existing cabinet, an existing refrigerator, an existing freezer, and existing heating unit, and the like. In this embodiment certain components, e.g., the security arrangement 20, may be retrofitted to the existing unit, and the local controller 18 (or, as discussed hereinafter, some other networked computer) may be in direct or indirect communication therewith. Accordingly, some or all of the components and/or features of the system 10 can be used or implemented in connection with an existing storage unit.

In another preferred and non-limiting embodiment, the storage unit 12 may have multiple accessible and/or controllable portions or areas. Accordingly, the storage unit 12 (or a portion thereof) may be refrigerated, heated, temperature-controlled, humidity-controlled, pressure-controlled, and the like. Such an arrangement is useful when certain types of samples or specimens S must be kept cold, warm, away from visible light, away from other specimens S, or otherwise in some controlled environment, e.g., a temperature-controlled environment, a humidity-controlled environment, a pressure-controlled environment, a shielded environment, and the like.

Another preferred and non-limiting embodiment of the storage unit 12 according to the present invention is illustrated in FIG. 3. As seen in this embodiment, the storage unit 12 is in the form of a cabinet 22, and the access point 14 is a door 24 that can be opened to access at least a portion of the internal area of the unit 12. In particular, the user U uses the handle 26 to open the door 24 to place a specimen S in or remove a specimen S from the storage unit 12. Further, in this embodiment, the storage unit 12 includes casters 28 or wheels in order to allow it to be mobile, and the local controller 18 is positioned on top of a body 30 of the storage unit 12. In addition, and in another preferred and non-limiting embodiment, the door 24 is primarily constructed from or at least partly constructed from glass in order to allow the user U to view the contents of the storage unit 12. Of course, as discussed above, the access point 14, e.g., door, drawer, and the like, can be opaque or shielded to protect the specimen from exposure to light. Further, the storage unit 12 or a portion thereof may be shielded in certain areas to avoid contact or potential contact between certain types of specimens S, e.g., radioactive specimens S, highly-reactive specimens S, contamination-prone specimens S, and the like.

With continued reference to FIG. 3, the storage unit 12 may have multiple shelves 32, hangers 34, or compartments 36. The shelves 32 can be used to support the containers C holding the specimens S within the storage unit 12, and the hangers 34 can be used to support various configurations of containers C, such as bags and other flexible containers that store fluid specimens S. In addition, the compartments 36 may also include handles 26 to either open a door 24 associated with each compartment 36. Of course, these compartments 36 may also be in the form of drawers that slide out to allow access to the contents therein. In addition, it is envisioned that these separate compartments 36 can be controlled to specific environmental conditions (as discussed above), such as refrigeration, heating, a specific temperature, a specific humidity, a specific pressure, and the like. Still further, the compartments 36 may be in the form of a manufactured structure to support specific types of specimens S or containers C. Again, the local controller 18 may effectively control both the storage unit 12, as well as any of the compartments 36 therein.

As further illustrated in FIG. 3, the storage unit 12 can be provided with a user interface unit 38. This user interface unit 38 can be attached to or integrated with the storage unit 12, and may be in direct or indirect, wired or wireless communication with the local controller 18, or some other controller or computer (as discussed hereinafter). This user interface unit 38 may include a display 40 and one or more input devices 42. The display 40 (as controlled by the local controller 18 or using its own processor) may be configured to allow for user interaction with the storage unit 12. Further, this user interface unit 38 may be in direct or indirect contact or communication with the security arrangement 20 in order to allow authorized access to the storage unit 12, whether a specially-manufactured storage unit 12 or a preexisting storage unit 12. This authorized access may be achieved through a variety of input devices 42, such as a radio frequency identification card, a magnetic strip identification, voice recognition, biometric input, keyboard-entered passwords or credentials, touch-screen entered passwords or credentials, and the like. In addition, this user interface unit 38 may allow for data input and/or output for managing and tracking the containers C and specimens S placed in or removed from the storage unit 12. Still further, this user interface unit 38 can be attached to or integrated with the storage unit 12 at a convenient location and position to allow for easy access to the user U.

In another preferred and non-limiting embodiment, the local controller 18 is programmed or configured to generate, receive, and/or transmit at least a portion of the storage unit data, at least a portion of the specimen data, message data, e-mail data, text data, voice data, status data, alert data, or any combination thereof. Accordingly, the local controller 18 can be used to directly or indirectly communicate useful data, messages, notifications, statuses, alerts, and the like throughout the system 10, such as to the users U of one or more user computers UC operating within the system 10 over a network N. Of course, it is envisioned that the user computer UC can be directly wired to or connected to the local controller 18 separate and apart from the user interface unit 38. Various exemplary wired and wireless connections are illustrated in the preferred and non-limiting embodiment of FIG. 2.

Still further, the local controller 18 may be programmed or configured to generate, receive, and/or transmit at least one of the following: (a) a notification relating to a specimen S custody chain; (b) a notification relating to access to the storage unit 12; (c) a notification that the storage unit 12 has been accessed; (d) a notification that the storage unit 12 has been accessed and one other action or inaction has been detected; (e) a notification that includes at least a portion of the storage unit data and/or the specimen data; (f) a notification that at least one specimen S has been placed in or removed from the storage unit 12; (g) a notification that a specimen S has been placed in or removed from the storage unit 12, and at least one other action or inaction has been detected; (h) a notification including time information relating to the specimen S; (i) a notification including results or test information relating to the specimen S, or any combination thereof.

Accordingly, the local controller 18 (or, as discussed hereinafter, another computer or controller) is able to generate, receive, and/or transmit (e.g., facilitate) a notification or message for delivery to one or more users U, such as through the user computers UC in the system 10. These notifications include important information relating to the life cycle management of the specimen S and can be used to track the location, related timing, and any other information or data relating to the storage unit 12 and/or specimen S at any point in the process. This allows any particular user U to quickly understand where the specimen S is in its life cycle and allows this user U to react accordingly.

With continued reference to FIG. 2, and in this preferred and non-limiting embodiment, one or more networked computers 44 are provided, and these networked computers 44 are in direct or indirect communication with the storage unit 12, such as through the local controller 18. Accordingly, this networked computer 44 may be programmed or configured to initiate one or more actions based upon the storage unit data, the specimen data, and/or any other data generated by or included within the system 10. The networked computer 44 may communicate directly or indirectly and wired or wirelessly with the storage unit 12, the local controller 18, another networked computer 44, the user computer UC, the user interface unit 38, or any other computer or appropriate device in the system 10. For example, and as further illustrated in FIG. 2, the user computer UC may take the form of a mobile device, such as a smart phone, a laptop, a pad computer, a notebook, a PDA, and the like. Accordingly, the useful information, such as the above-discussed notifications, statuses, alerts, messages, e-mails, texts, voice data, and the like, can be delivered to any user U utilizing any device in the system 10, such as a device operating on or with the network N. Accordingly, the networked computer 44 may take the place of one or more of the functions of the local controller 18 (or the controller 18, itself), and generate, receive, and/or transmit any of the above-discussed data and information. Therefore, the functions and features of the local controller 18 and the networked computer 44 can be coextensive, separated, and/or segregated in any preferable manner. Accordingly, the networked computer 44 may control any of the components attached to, included with, or integrated with the storage unit 12.

In another preferred and non-limiting embodiment, the networked computer 44 (and/or the local controller 18) includes a database 46 at least partially populated with any of the data and information described above in detail. Further, this database 46 can be maintained in a networked computer 44 in any described manner, such as where the networked computer 44 is a virtual computer, a cloud-based system, a server, a personal computer, a mobile device, and/or any other appropriate computing platform or architecture. In addition, this database 46 can be stored in multiple locations in order to ensure redundancy and for use in certain situations, e.g., an emergency situation where the primary networked computer 44 or server is inoperable or inaccessible. In this manner, the system 10 can continue to operate in any situation and effectively allow for the management of specimens S throughout their life cycle, which is especially important in a hospital or clinical setting.

In a further embodiment, and as also illustrated in FIG. 2, multiple storage units 12 are included in the system 10. Preferably, these storage units 12 are placed (or may already be present) at various locations throughout the hospital or lab, such as the functional locations where the specimen S “stops” during its life cycle. Of course, it is further envisioned that multiple storage units 12 can be located at the same location, where each storage unit 12 is directed to a specific type of specimen S or satisfies the environmental needs of specific types of specimens S.

In another preferred and non-limiting embodiment, and as illustrated in FIG. 4, the identification device 16 may be in the form of a tag that can be permanently or removably attached to the container C or specimen S. In this embodiment, the identification device 16 may include patient data 48, such as the patient's name or identification number, specimen-related data 50, such as the date and time of collection or some stage of delivery, and/or an identification of the specimen S, handwritten data 52, which will allow a user U to make notes or otherwise provide information that will be associated with the specimen S, and tracking data 54, such as a barcode, a QR code, a transponder (for use in a radio frequency identification system), or any other scannable, trackable, or recognizable code associated with the specimen S or patient

In a still further preferred and non-limiting embodiment, the present invention is directed to a computer-implemented method for tracking at least one specimen S. This method is implemented on one or more of the computers or computing devices discussed above. In this embodiment, this method includes: detecting the entry of the at least one specimen S into the tracking system 10; determining specimen data at least partially associated with the at least one specimen S, wherein the specimen data at least partially comprises at least one of the following: patient data, time data, specimen collection data, specimen purpose data, specimen collector data, specimen collection location data, health care provider data, laboratory data, procedure data, test data, body data, surgery data, storage unit identification data, location data, custody data, results data, compliance data, inventory data, authorization data, access data, user data, status data, alert data, or any combination thereof; and based at least partially on at least a portion of the specimen data, generating, receiving, and/or transmitting at least one of the following: at least a portion of the specimen data, message data, e-mail data, text data, voice data, status data, alert data, or any combination thereof.

In a still further preferred and non-limiting embodiment, the system 10, arrangement, and/or method include one or more user interface units 38 and/or input devices 42 positioned throughout the location, e.g., the hospital, the laboratory, the building, the campus, and the like. These interface units 38 and/or input devices may be considered “waypoints” as the specimen S moves around the location, e.g., from room to room, from department to department, from building to building, from location to location, and the like. The user U would “log” the specimen S or otherwise interact with the user interface unit 38 and/or input device 42 such as to identify the location of the specimen S at a particular time (and, optionally, the identification of the user U), which facilitates further tracking of the specimen S during its lifecycle. These “waypoints” may be in the form of a standalone user interface unit 38 (e.g., a kiosk), a standalone input device 42 (e.g., an RFID tag reader, a scanner, a QR reader, and the like), and/or associated with a specified storage unit 12. Similarly, the user U may use his or her mobile device as a “waypoint” at certain times, which may associate the user U location (i.e., the specimen S location) with a specific location and user U. Still further, the various rooms in the building may be equipped with a door scanner (as the input device 42) that passively or actively reads any of the identification devices 16 as they are moved through the door or opening. In addition, these “waypoints” may be included and tracked or controlled as part of the system 10, or some other existing hospital/health care system or process. Accordingly, this would provide a further level of specimen S management and tracking, which does not require the direct use of the above-described storage units 12 (although these storage units 12 could also be used as or considered a “waypoint” within the context of this embodiment).

In a still further preferred and non-limiting embodiment, the method includes: detecting access to at least one storage unit 12; detecting the addition or removal of at least one specimen S from the storage unit 12; determining storage unit data at least partially associated with the storage unit 12 and/or specimen data at least partially associated with the specimen S; and generating, receiving, and/or transmitting at least one of the following: at least a portion of the storage unit data, at least a portion of the specimen data, message data, e-mail data, text data, voice data, status data, alert data, or any combination thereof.

These methods further illustrate the end-to-end life cycle management and control of a specimen S and/or container C associated with a patient.

In another preferred and non-limiting embodiment, the container C is in the form or size of a sample cup, and the identification device 16 is a label that is applied to the container C. It should be recognized that the container may be larger, or could be in any useful form, such as a bag, a test tube, and the like. In this embodiment, the container C is labeled with the identification device 16, which is in the form of a radio frequency identification label, which may be built or programmed at the point of manufacture. Accordingly, the container C can be manufactured with an integrated identification device 16, but, as discussed above, this identification device 16 (or tag) may be placed on post-manufacture with adhesive or the like at the location. As discussed, the container (or a portion of the identification device 16 (or label)) may include an area for the clinician to hand-write information about the specimen S collected, such as the patient name, the time, the specimen collected, the purpose, who collected it, where it was collected, and the like. In this embodiment, after the specimen S has been collected, it is placed into the container C, and the container C is then closed and/or sealed. This container C will then be transported to the above-discussed storage unit 12, which, in this embodiment, is in the same department or location as the place where the specimen S was collected, for convenience and record-keeping purposes.

In another exemplary embodiment, the storage unit 12 is in the form of a cabinet 22 and includes an electrically-operated lock (as part of the security arrangement 20), and this lock can be actuated through application software, for example loaded on the local controller 18 to provide physical security. This lock can be “defeated” with a key-operated manual override, if necessary. Further, the storage unit 12 includes multiple shelves 32, and may also be configured to store ambient temperature samples. Of course, this storage unit may be modified (or another storage unit 12 used) that includes refrigeration, heating, or other environmental controls to store samples that require refrigeration, freezing, heating, or other special environmental conditions.

With respect to refrigeration, and in one exemplary embodiment, and since a hospital normally has already invested in a refrigerator and/or freezer, the local controller 18, the security arrangement 20, and/or the user interface unit 38 can be retrofitted on an existing refrigerator (i.e., an existing storage unit 12). Accordingly, the system 10 may use a lock controlled by any of the components or units in the system 10 in connection with both specially-manufactured and existing storage units 12. In this example, the specially-manufactured storage unit 12, the local controller 18, the networked computer 44, and/or the user interface unit 38 will also control access to the existing storage unit, e.g., a refrigerator or freezer, by facilitating the locking and unlocking of the lock at the appropriate times in a work flow.

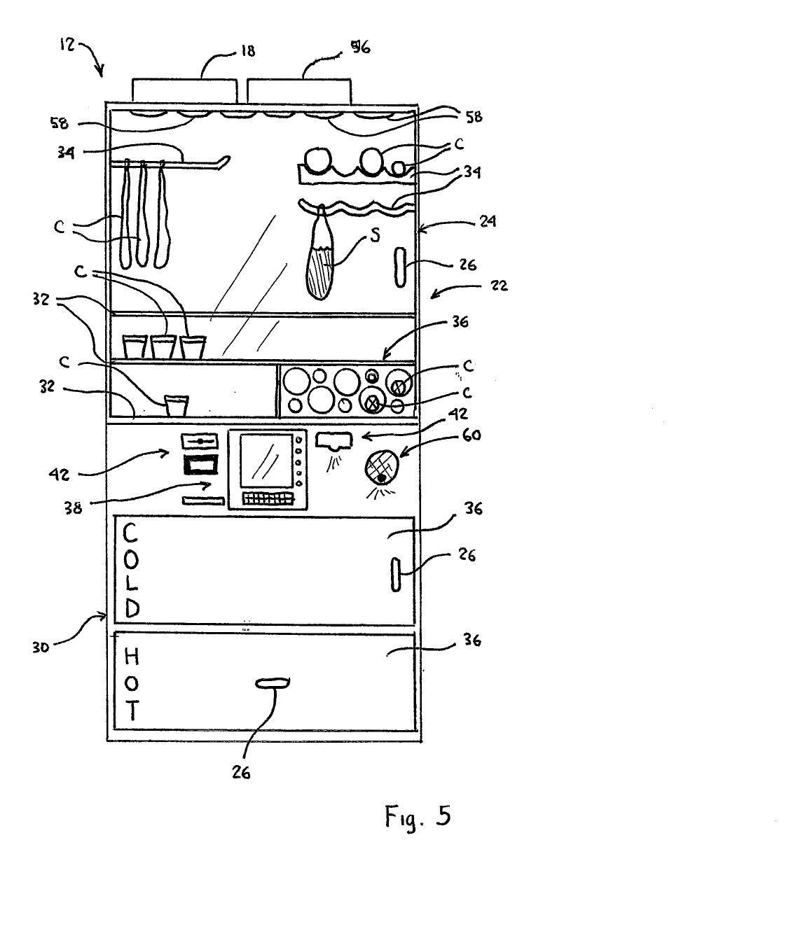

In another preferred and non-limiting embodiment, and as illustrated in FIG. 5, the storage unit 12 may be lit by one or more lights 58, such as an arrangement of overhead lighting. This lighting arrangement (or lights 58) may be controlled through application software on the local controller 18. Accordingly, the lights 58 may include a series of colored, e.g., red, lights, or blinking lights, and the like, which are turned on to indicate a problem within the storage unit 12. In addition, these lights 58 may indicate any specific issue with the specimens S positioned in the storage unit 12. Still further, these lights 58 may indicate an issue, problem, status, state, and/or alert in connection with any of the components of the storage unit, e.g, the controlled components, such as the user interface unit 38, the controlled compartments 36, and the like. In this embodiment, the local controller 18 is positioned above the body 30 of the storage unit 12, and it is envisioned that any of the electronics 56 associated with the storage unit 12 can be positioned or situated above the storage unit 12 (preferably in a sealed chassis, thereby providing radio frequency shielding and allowing the storage unit 12 interior to be completely open for effective use).

With continued reference to the preferred and non-limiting embodiment of FIG. 5, the user interface unit 38 is provided, and includes a display 40, multiple input devices 42, and at least one output device 60. This output device 60 may be in the form of a speaker and/or visual indicator to provide immediate information regarding the specimens S in the storage unit 12, or any component of the storage unit 12, itself. For example, the above-discussed alert data may be provided in an audio or visual format, as provided by the output device 60.

With continued reference to this embodiment, the storage unit 12 is accessed either with a pin number, a user account/password combination, validation of a user U with a radio frequency embedded identification card (typically HID or barcode), and the like. Additionally, biometric information, such as finger and thumb print, eye-iris and retina scanning, and the like can be used, as provided by the appropriate input devices 42 of the user interface unit 38. In additional, the storage unit may include a barcode reader, which can be used to read data including patient EMR or account numbers, which represent unique identifiers of the patient and/or the visit.

In another preferred and non-limiting embodiment, the storage unit 12 includes multiple shelves 32 that include, are formed with, or are integrated with one or more antennae. These antennae are designed for use in connection with radio frequency identification (RFID) tags (i.e., the identification devices 16). In one preferred and non-limiting embodiment, these tags are HF ISO tags, and in other embodiments, they may be UHF, EPC, and any other types of RFID tags. The location of the antennae may be optimized such that the maximum number of specimens S can be stored in the storage unit 12.

In one preferred and non-limiting method of operation, the specimens S are collected and placed into the containers C (which may be placed into one or more other containers C). These containers C are then brought to the storage unit 12 location, and an authorized user U will access the storage unit 12 using his or her credentials, as verified by the system 10. The user U then selects the patient from a list that includes all patients in the department, and possibly the hospital. Selection may also occur through the use of a barcode associated with a patient. In addition, an ADT interface or scheduling interface may be utilized. Still further, the user interface unit 38 can be used and receive any of the information discussed above, such as the patient name, doctor, procedure, and related data.

In this embodiment, and at this point, the hospital may elect that the user U enter detailed information about the specimen S beyond the patient name. This may include, but is not limited to, the type of specimen, time of collection, test requested, clinician requesting (since the person placing the specimen S in the storage unit 12 may be different than the clinician requesting the test), which part of the body the specimen S was collected from, whether this is an in-surgery or out-of-surgery situation, where the collection occurred, who collected it, and the like. The various data, information, and fields may be configured through application software loaded on the local controller 18, the user interface unit 38, and/or the networked computer 44, and is dependent upon the needs of the hospital or healthcare setting.

Next, and in this embodiment, the door 24 of the storage unit 12 will unlock. The authorized user U will then place the specimen S into the storage unit 12 and close the door 24. As this happens, the information and data entered by the user, e.g., at the user interface unit 38, will be associated with the identification device 16 of the container C. The system 10 will also store the time the specimen S was positioned or inserted into the storage unit 12. As discussed, the storage unit 12 may be refrigerated, heated, or otherwise controlled for a variety of environmental conditions.

In the situation where the storage unit 12 is not refrigerated, but refrigeration is required for storage of the specimen S, the above-discussed lock may be used in connection with a refrigerated unit, and this lock may be controlled by an adjacent or local storage unit 12, the local controller 18, the user interface unit 38, the networked computer 44, and the like. In addition, a kiosk, i.e., a user interface unit 38, may be placed on or near and integrated with a security arrangement 20 for access to a refrigerated unit that is a pre-existing unit. In this situation, the authorized user U accesses the controlling storage unit 12 and/or the kiosk (the user interface unit 38), selects a patient and enters the required information. At this point, the system 10 waits for the user U to scan the identification device 16 on the container C on an externally-mounted RFID antenna (i.e., input device 42) on the storage unit 12 or user interface unit 38. The system 10 then detects the RFID number from the identification device 16 and associates the information entered regarding the patient. The system 10 then unlocks the electronically-controlled lock mounted to the refrigerator, freezer, controlled environment, etc., and the lock is designed such that when the door opens, it sends a signal of this occurrence back to the system 10. The user U opens the door and places the specimen S into the controlled refrigerator, freezer, controlled unit, and the like, and then closes the door. The user U can then either manually log out from the system 10, or the system 10 will log out automatically after a certain period of time.

Continuing with this embodiment, if the user U does not open the door on the remote-controlled lock on the refrigerator, the system 10 may also detect this activity/inactivity. If this occurs, the system 10 will alert the user U to place a specimen S into the storage unit 12, refrigerator, separate unit, and the like, or cancel the transaction. If, after a specified period of time lapses and no sample or specimen S has been placed in the storage unit 12, refrigerator, and the like (or is not detected to be by the system 10), and the door is not opened, the system 10 will send an alert to supervisors of the issue, and log out. Generally, this procedure can be used in connection with any of the storage units 12 discussed above, as well as with an existing (but controlled) storage unit 12, such as an existing refrigerator, an existing freezer, an existing heated unit, an existing environmentally-controlled unit, and the like. Generally, if the user U does not follow the required instructions, does not input the correct information, does not place a specimen S in the unit after entering the information, does not open the door after a set period of time, and/or engages in any other detectable activity or inactivity, the system 10 can control the unit and/or send appropriate notifications to other users U in the system 10.

Continuing with the above-discussed embodiment, and after the specimen S is placed in the storage unit, the system 10 records the time of specimen S storage and immediately sends a signal to the testing department, e.g., the pathology department, in the hospital or external testing organization. The signal may be in the form of a notification, such as an e-mail, a text, a page, an automated call, an entry into the hospital system, an entry into the testing system, and the like. This eliminates the need for the clinician in the hospital to call the department. It also eliminates the need for the testing department to spend time receiving a call. Accordingly, the testing department will have an electronic inventory of all specimens S awaiting pickup. The dispatch of support staff can occur in an organized fashion to the rest of the hospital. Any time that a specimen S has sat in a storage area longer than a predetermined period, the system 10 may also send an alert to the testing department supervisor, as well as the supervisors in the department requesting the test.

Continuing with this embodiment, the support staff dispatched to pick-up the specimen S may have preauthorized access. The staff will log in to the storage unit 12, refrigerator, freezer, controlled unit, and the like. This user U will be shown a screen in which they can select to remove samples for transport. The staff will then remove all the items for transport and place them into their own container or holder. It is further envisioned that this user U container or holder may also be in the form of or include an identification device 16 that enables the specimen S to be tracked as to where and when it was placed in this user U container. By using RFID scanning, the RFID-enabled storage unit 12, whether in the form of a specially-manufactured or modified existing storage unit 12, will detect the items that are removed for transport, who removed these items, and provide a time-stamp for the transaction. In the case of a modified existing storage unit 12, e.g., a modified existing unit that is controlled by an external lock, the system 10 may also unlock the door, and the support staff will wave the identification device 16 over an external RFID reader (i.e., an iput device 42) at the user interface unit 38 to capture the items removed from the unit. Again, any information can be associated with this transaction and tracked in connection with the specimen S, storage unit 12, or any other component in the system 10. The support staff will then transport the items to the pathology/testing department, and/or some commercial testing location. At the testing location, it is envisioned that the specimens S can also be logged in as “received” through the use of another storage unit 12, some separate unit, an RFID antenna pad, and the like.

In one preferred and non-limiting embodiment, the specimens S are added and/or removed only after an authorized user U accesses the storage unit 12, and selects a patient from a list that includes all potential related patients. It is envisioned that an ADT interface feed may be used. Further, the system 10 includes a variety of interfaces for receiving the specimen data and/or storage unit data. Still further, the system 10 provides for dynamic and automated updating of inventories, storage unit data, specimen data, and the like. Therefore, the user U is always presented with real-time data. In addition, the link between the patient and the specimen S is preferably maintained in perpetuity, such that clinicians can be alerted if some issue arises with a specimen S and/or some change with the patient.

In another preferred and non-limiting embodiment, and as illustrated in FIG. 5, the local controller 18 and electronics 56 are positioned at the top of the storage unit 12, but may be placed at other positions outside the storage unit 12, thus freeing the interior for maximum storage of specimens S. The door 24 or front of the storage unit 12 may be glass or some other transparent material, but may also be manufactured from an opaque material if storing specimens S that are not supposed to be exposed to direct light. In addition, the networked computer 44 may also be positioned on top of the storage unit 12. By positioning the local controller 18, the electronics 56, and/or the networked computer 44 at the top of the storage unit 12, the storage unit 12 is easily maintained. This also allows for retrofitting the local controller 18 and/or the networked computer 44 for use in connection with a separate, controllable unit, e.g., an existing refrigerator, freezer, heater, and the like. Of course, it is envisioned that the local controller 18 and/or the networked computer 44 can be in the form of a server that is positioned in a separate area, such as a server farm/rack of the hospital, and connected through the hospital network N.

As discussed, and in this embodiment, the user interface unit 38 provides a display 40 and various input devices 42 to facilitate effective management and interaction with a user U. The display 40 may be a touch-screen, and this unit 38 may be attached to the door 24, i.e., swing when the door 24 is opened, to provide full access to the interior of the storage unit 12, as well as convenient access to the display 40.

In this preferred and non-limiting embodiment, the input device 42 includes an RFID reader or barcode reader and/or any appropriate input device, e.g., for an HID card, a magnetic strip reader, and the like. These input devices 42 may be integrated with the storage unit 12 to permit only authorized access and/or other beneficial functionality, e.g., patient selection. In addition, the user interface unit 38 may include an input device 42 in the form of a keyboard or buttons that provide for direct input or assigned button-type input, e.g., “find specimen”, “cabinet inventory”, and the like. Such specified and dedicated buttons or selections may allow a user U to understand information or data pertaining to the storage unit 12 and/or the system 10 without requiring authorized access to the contents of the storage unit 12.

As discussed, the storage unit 12 may include lights 58 and/or other output devices 60 to provide audio or visual alerts. This facilitates a quick understanding of a specified issue regarding the storage unit 12 or the specimens S stored therein. Again, these lights 58 may be differently colored, blinking, and the like, in order to indicate a specific issue. In addition, the lights 58 may provide internal lighting to the storage unit 12.

In another preferred and non-limiting embodiment, the specimens S are held securely, whether on the shelves 32, on or supported by hangers 34, on or within compartments 36, and the like. The storage unit 12 may be manufactured from a secure and easy-to-clean material, and the security arrangement 20 may be in the form of a lock manufactured to fit a variety of refrigerators, freezers, controllable units, cabinets, and other containers in which specimens S can be stored. In addition, it is envisioned that the external lock may contain a sensor to know when the door is physically opened and closed by an authorized user U. Still further, a single storage unit 12 may have multiple areas that are individually locked, such that one or more of these separate locked areas may only be accessed by an authorized user U.

In another preferred and non-limiting embodiment, and as discussed above, the input device 42 may be in the form of an RFID antenna pad, which is an external mechanism without security features, and which quickly enables the users U in the pathology department to mark specimens S as “received”. An RFID antenna may be attached to the user interface unit 38 or some other kiosk programmed or configured with the application software to receive and track the specimen S. In this embodiment, the support staff that acquired the specimen S from the hospital departments will identify themselves at the user interface unit 38, interact with a button to say that they are scanning specimens S, and then scan each specimen S into the inventory at the pathology/testing department. The system 10 would then time-stamp each item as it enters the system 10, together with the user U that placed them into the inventory of the pathology/testing department and/or storage unit 12. Also, in this embodiment, it is envisioned that the local controller 18 and/or the networked computer 44 maintains records of all transactions within the system 10. In addition, the local controller 18 and/or the networked computer 44 manages the interfaces with other systems for data feeds, such as patient and/or procedure information.

In another preferred and non-limiting embodiment, and as discussed above, the system 10 includes various notifications and/or alerts built in to notify staff of potential issues in the custody chain of the specimen S. For example, at the point of entry of a specimen S into a storage unit 12, e.g., a refrigerator with a lock, if the user U opens the door of the refrigerator without scanning the container C at the input device 42, the system 20 will first alert that user U on the display 40, together with providing an audible alert. If no action is taken thereafter, and after a predetermined amount of time, an alert, such as an e-mail, text, and the like, will be transmitted to the user U that accessed the storage unit 12, as well as to the department supervisors. Similarly, if the support staff from the pathology department that have come to pick up the specimens S open the storage unit 12, and then do not scan the container C at the input device 42, a similar alert will go out to that user U as well as the department supervisors and pathology/testing department supervisors. This alert ensures compliance with various rules and regulations associated with the system 10.

Still further, and in another preferred and non-limiting embodiment, time-based escalation alerts may be used. If a specimen S is placed into a storage unit 12, refrigerator, or other controlled unit, and a predetermined amount of time passes before pick up, an alert will be transmitted to the requesting clinician, department supervisors, and/or pathology supervisors. This ensures that specimens S are picked up in a time span agreeable between department and pathology supervisors. A similar alert to the same individuals may also go out if a specimen S is in transit from the department to the pathology/testing labs if a certain period of time lapses.

In another preferred and non-limiting embodiment, an additional alert notifying the requesting clinician that the results of the test are ready may be implemented and transmitted. By either manually entering the results into the system 10, or integrating the system 10 with the pathology test system, the present system can immediately alert the requesting clinician. This alert may include the results of the test, or for privacy purposes of the hospital, just a notification that the test and/or results are ready. One function of this is to enable the clinician to engage in other work, and only focus on the specified specimen S when the results are ready. Accordingly, the clinician does not have to constantly call the pathology department or constantly check the pathology testing system on the computer to see if the results are ready. In another preferred and non-limiting embodiment, the present invention provides for appropriate compliance record keeping based upon the storage unit data and/or the specimen data. For example, the system 10 allows for the generation, receipt, and/or transmission of certain information required for compliance tracking, such as when the specimen S was acquired, when the specimen S was placed into storage, whether the specimen S was placed into cold storage, how long he specimen S was out of cold storage, how long it took the specimen S to be picked up after being placed into storage, whether any alerts or notifications were provided, how long the specimen S spends in transit, how long the specimen S spends in the pathology/testing department before being tested, and the like. This eliminates the need for paper record-keeping, thus reducing the resultant human errors. In addition, the system 10 provides for technician and clinician monitoring and tracking, as well as work flow time stamps and tracking.

In another preferred and non-limiting embodiment, the system 10 allows for individual specimen S identification, such as through the use of field-applied identification devices 16 (e.g., RFID tags) on the containers C, whether manufactured with the container C or applied in a post-manufacture process. Information and data may be written to and/or stored on the identification device 16 relating to the specimen S. Alternatively, the identification device 16 may act as a pointer to information stored in a separate database. In addition, these identification devices may be programmed or configured for any desired purpose within the system 10.

In this manner, the presently-invented specimen management system, arrangement, and method provide a variety of beneficial functionalities for specimen management and tracking For example, in one embodiment, the present invention provides for loss prevention through the identification of the nurse/clinician that stores/removes the specimen S. For example, an alert may be issued if a specimen S is removed from or not appropriately placed in the system 10, e.g., the storage unit, after a specified period of time. In addition, an alert may be issued if an item or specimen S is in transit for a specified period of time. In addition, the security arrangement 20 allows the storage units 12 and/or the other related and controlled units to be locked and only provide authorized and recorded access.

The presently-invented system, arrangement, and method also improve patient safety. For example, the notifications or messages may be sent via e-mail to a variety of user computers UC, including mobile or handheld devices. In addition, the system 10 ensures that specimens S are labeled and identified correctly, that these specimens S are tested in an appropriate period of time, and that these specimens S are not lost, thereby eliminating the need to acquire a specimen S from a patient more than one time. These alerts or messages/notifications may be issued in real time, and the system 10 may also provide reports of history, usage, storage unit information, specimen information, or any of the data information discussed above. In addition, the closed and secure design of the storage unit 12 protects difficult-to-acquire specimens S.

Another benefit of the presently-invented specimen management system, arrangement, and method is the improvement to work force management. For example, the system 10 facilitates compliance monitoring of protocols required for effective handling of specimens S, and provides time-based measures for work force efficiencies. In addition, it is envisioned that the system 10 can be used to find the specimen S or other medical equipment. For example, the user U can use a “find specimen” inquiry to locate a specimen S, and a requesting clinician can see the current status of specimens S without having to call and waste the time of the pathology/testing department personnel. In addition, the clinician and staff can see whether a specimen S is in a department storage unit 12, in transit, at the pathology/testing department, and whether the testing has started, whether it is complete, and whether the test results are ready.

Another benefit relates to the management tools provided by the system 10. As discussed above, the alerts or notifications can be sent to the managers in real time, and these messages may be delivered to the inbox or user computers UC, or other designated e-distribution locations. In addition, reports can be generated and provided to the users U indicating actions during storage, retrieval, transit, testing, and the like.

Accordingly, the present invention provides a specimen management system, arrangement, and method that facilitates the effective tracking and management of multiple specimens S over their life cycle.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A specimen management system, comprising:
at least one storage unit comprising an at least partially enclosed structure having at least one access point configured to provide access to the at least one storage unit, wherein the at least one storage unit is at least partially associated with storage unit data and configured to receive and store at least one specimen, which is associated with at least one identification device associated, configured, or programmed with specimen data at least partially associated with the at least one specimen; and
at least one local controller programmed or configured to generate, receive, and/or transmit at least a portion of at least one of the storage unit data and the specimen data, wherein the at least one local controller is programmed or configured to initiate at least one action based at least partially on at least a portion of at least one of the storage unit data and the specimen data.

2. The specimen management system of claim 1, wherein the at least one specimen is at least partially positioned in a container, and the at least one identification device is associated with the container.

3. The specimen management system of claim 2, wherein the at least one identification device is a radio frequency identification tag attached to at least a portion of the container.

4. The specimen management system of claim 1, wherein at least one of the storage unit data and the specimen data at least partially comprises at least one of the following: patient data, time data, specimen collection data, specimen purpose data, specimen collector data, specimen collection location data, health care provider data, laboratory data, procedure data, test data, body data, surgery data, storage unit identification data, location data, custody data, results data, compliance data, inventory data, authorization data, access data, user data, status data, alert data, or any combination thereof.

5. The specimen management system of claim 1, wherein the at least one storage unit comprises a security arrangement configured to provide selective access to the at least one storage unit through the at least one access point.

6. The specimen management system of claim 5, wherein the at least one local controller is in communication with the at least one security arrangement and programmed or configured to unlock the at least one access point based at least partially upon user interaction with the at least one storage unit.

7. The specimen management system of claim 1, wherein at least a portion of the at least one storage unit is at least one of the following: refrigerated, heated, temperature controlled, humidity controlled, pressure controlled, or any combination thereof.

8. The specimen management system of claim 1, wherein the at least one local controller is in communication with at least one user interface unit attached to or integrated with the at least one storage unit.

9. The specimen management system of claim 1, wherein the at least one local controller is programmed or configured to generate, receive, and/or transmit at least one of the following: at least a portion of the storage unit data, at least a portion of the specimen data, message data, e-mail data, text data, voice data, status data, alert data, or any combination thereof.

10. The specimen management system of claim 1, wherein the at least one local controller is programmed or configured to generate, receive, and/or transmit at least one of the following:
(a) a notification relating a specimen custody chain;
(b) a notification relating to access to the at least one storage unit;
(c) a notification that the at least one storage unit has been accessed;
(d) a notification that the at least one storage unit has been accessed and at least one other action or inaction has been detected;
(e) a notification comprising at least a portion of at least one of the storage unit data and the specimen data;
(f) a notification that at least one specimen has been placed in or removed from the at least one storage unit;
(g) a notification that at least one specimen has been placed in or removed from the at least one storage unit and at least one other action or inaction has been detected;
(h) a notification including time information relating to the at least one specimen; and
(i) a notification including results or test information relating to the at least one specimen;
or any combination thereof.

11. The specimen management system of claim 1, further comprising at least one networked computer in direct or indirect communication with the at least one storage unit and configured to receive at least a portion of at least one of the storage unit data and the specimen data; wherein the at least one networked computer is programmed or configured to initiate at least one action based at least partially on at least one of the storage unit data and the specimen data.

12. The specimen management system of claim 11, wherein the at least one networked computer is configured to generate, receive, and/or transmit at least one of the following: at least a portion of the storage unit data, at least a portion of the specimen data, message data, e-mail data, text data, voice data, status data, alert data, or any combination thereof.

13. A specimen management system, comprising:
at least one storage unit comprising an at least partially enclosed structure having at least one access point configured to provide access to the at least one storage unit, wherein the at least one storage unit is at least partially associated with storage unit data and configured to receive and store at least one specimen, which is associated with at least one identification device associated, configured, or programmed with specimen data at least partially associated with the at least one specimen; and
at least one networked computer in direct or indirect communication with the at least one storage unit and configured to receive at least a portion of at least one of the storage unit data and the specimen data, wherein the at least one networked computer is programmed or configured to initiate at least one action based at least partially on at least a portion of at least one of the storage unit data and the specimen data.

14. The specimen management system of claim 13, wherein the at least one storage unit further comprises at least one local controller programmed or configured to generate, receive, and/or transmit at least a portion of at least one of the storage unit data and the specimen data, wherein the at least one local controller is programmed or configured to initiate at least one action based at least partially on at least one of the storage unit data and the specimen data.

15. The specimen management system of claim 14, wherein the at least one local controller is programmed or configured to generate, receive, and/or transmit at least one of the following: at least a portion of the storage unit data, at least a portion of the specimen data, message data, e-mail data, text data, voice data, status data, alert data, or any combination thereof.

16. The specimen management system of claim 13, wherein the at least one networked computer is configured to generate, receive, and/or transmit at least one of the following: at least a portion of the storage unit data, at least a portion of the specimen data, message data, e-mail data, text data, voice data, status data, alert data, or any combination thereof

17. The specimen management system of claim 13, wherein the at least one networked computer is programmed or configured to generate, receive, and/or transmit at least one of the following:
(a) a notification relating a specimen custody chain;
(b) a notification relating to access to the at least one storage unit;
(c) a notification that the at least one storage unit has been accessed;
(d) a notification that the at least one storage unit has been accessed and at least one other action or inaction has been detected;
(e) a notification comprising at least a portion of at least one of the storage unit data and the specimen data;
(f) a notification that at least one specimen has been placed in or removed from the at least one storage unit;
(g) a notification that at least one specimen has been placed in or removed from the at least one storage unit and at least one other action or inaction has been detected;
(h) a notification including time information relating to the at least one specimen;
(i) a notification including results or test information relating to the at least one specimen;
or any combination thereof.

18. The specimen management system of claim 1, wherein the at least one networked computer comprises at least one database at least partially populated with at least one of the following: patient data, time data, specimen collection data, specimen purpose data, specimen collector data, specimen collection location data, health care provider data, laboratory data, procedure data, test data, body data, surgery data, storage unit identification data, location data, custody data, results data, compliance data, inventory data, authorization data, access data, user data, message data, e-mail data, text data, voice data, status data, alert data, or any combination thereof.

19. The specimen management system of claim 1, further comprising a plurality of storage units, each having at least one local controller programmed or configured to control at least one function of the at least one storage unit, wherein the plurality of storage units are in direct or indirect communication over a network.

20. A computer-implemented method for tracking at least one specimen in a tracking system, comprising:
on at least one computer having a storage medium having stored thereon program instructions, which, when executed by a processor of the computer, cause the processor to:
detect the entry of the at least one specimen into the tracking system;
determine specimen data at least partially associated with the at least one specimen, wherein the specimen data at least partially comprises at least one of the following: patient data, time data, specimen collection data, specimen purpose data, specimen collector data, specimen collection location data, health care provider data, laboratory data, procedure data, test data, body data, surgery data, storage unit identification data, location data, custody data, results data, compliance data, inventory data, authorization data, access data, user data, status data, alert data, or any combination thereof; and
based at least partially on at least a portion of the specimen data, generate, receive, and/or transmit at least one of the following: at least a portion of the specimen data, message data, e-mail data, text data, voice data, status data, alert data, or any combination thereof.
